(19) United States
(12) Patent Application Publication
(10) Pub. No.: US 2019/0351181 A1
(43) Pub. Date: Nov. 21, 2019

(54) CATHETER ARRANGEMENT AND METHOD FOR PRODUCING SUCH A CATHETER ARRANGEMENT

(71) Applicant: B. BRAUN MELSUNGEN AG, Melsungen (DE)

(72) Inventors: EGBERT DUBIELZIG, Fritzlar (DE); OLIVER KAHLEN, Gudensberg (DE)

(21) Appl. No.: 16/411,538

(22) Filed: May 14, 2019

(30) Foreign Application Priority Data
May 16, 2018 (DE) 102018207642.1

(51) Int. Cl. A61M 25/00; A61B 5/00

(52) U.S. Cl. CPC A61M 25/0026; A61B 5/6852; A61M 25/001; A61M 25/008; A61B 2562/168; A61B 2562/12; A61B 2562/0247

(57) ABSTRACT

A catheter arrangement includes a catheter tube with a lumen. The lumen has a proximal end, a distal end, and at least one supply line. The supply line has a distal end and a proximal end. During a method of production, the distal end of the supply line is connected in a fluid-conducting manner to the proximal end of the lumen. The fluid-conducting connection between the supply line and the lumen can be brought about by a fluid-tight joining connection between the distal end of the supply line and the proximal end of the lumen. The catheter arrangement can be used in infusion therapy.

### RELATED APPLICATION

This application is related to and claims the benefit of priority of German Application No. 10 2018 207 642.1, filed May 16, 2018, the contents of which is incorporated by reference herein in its entirety.

### FIELD

The invention relates to a catheter arrangement comprising a catheter tube with at least one lumen, wherein the lumen has a proximal end and a distal end, and at least one supply line, wherein the supply line has a distal end and a proximal end, and wherein the distal end of the supply line is connected in a fluid-conducting manner to the proximal end of the lumen.

The invention also relates to a method for producing such a catheter arrangement.

### BACKGROUND

A catheter arrangement of this type is generally known in the sphere of medical technology and is provided in the form of a multi-lumen venous catheter for use in infusion and/or transfusion therapy. The known catheter arrangement has a catheter tube, through which a first lumen and a second lumen, which each have a distal end and a proximal end, are extended. The distal ends of the lumina are arranged in a portion of the catheter tube that, in a correctly applied state of the catheter arrangement, is introduced into, for example, a patient's vein. By contrast, in the applied state of the catheter arrangement, the proximal ends of the lumina are arranged outside the patient's body. Furthermore, a first supply line and a second supply line each having a proximal end and a distal end are provided. The first supply line is assigned to the first lumen. The second supply line is assigned to the second lumen. The proximal ends of the supply lines can each be provided in a basically known manner with a connection element for the fluid-conducting connection to an infusion container or the like. The distal ends of the supply lines are connected in a fluid-conducting manner to the proximal end of the respectively assigned lumen. Two channels which are separate from each other are thereby formed. Said channels extend from the proximal end of the respective supply line as far as the distal end of the lumen assigned to the respective supply line. The channels of the catheter arrangement that are separated from each other in such a manner permit, for example, simultaneous administration of incompatible medical fluids, with mixing of the fluids within the catheter arrangement being avoided.

In addition, the known catheter arrangement has a connecting unit, which is manufactured as a plastics injection-moulded component, for the fluid-conducting connection of the supply lines to the respective lumen. Said connecting unit is also referred to as a channel separator. The connecting unit is arranged between the distal ends of the supply lines and the proximal ends of the lumina and is injection moulded both onto the supply lines and onto the catheter tube. The connecting unit has two connecting channels which are arranged next to each other and are extended separately from each other. The distal ends of the supply lines and the proximal ends of the lumina lead on opposite end sides of the connecting unit into the connecting channels. The connecting channels to this extent each form a fluid-guiding portion of the channels, which are separate from each other, of the catheter arrangement.

In order to produce the known catheter arrangement, a supporting element in the form of what is referred to as an insert-moulding needle is in each case introduced into the proximal end of the first lumen and into the proximal end of the second lumen. Furthermore, the supply lines are pushed at their respective distal end onto the insert-moulding needle of the respectively assigned lumen, wherein the distal ends of the supply lines and the proximal ends of the lumina are spaced apart from one another. Furthermore, the arrangement in such a way is placed into an injection mould and insert moulded in the region of the insert-moulding needles with a thermoplastic. The insert-moulding needles are subsequently removed and the connecting unit found in such a manner is removed from the injection mould. The insert-moulding needles serve firstly for supporting the lumina and the supply lines since they would otherwise collapse during the plastics injection moulding because of pressure. Secondly, the insert-moulding needles form negative shapes for the respective connecting channel which extends between the distal end of the supply line and the proximal end of the assigned lumen.

During the production of the connecting unit by means of plastics injection moulding, manufacturing errors, for example shrinkage cavities, stress cracks or an incomplete formation, can occur. As a result, leakages in the fluid-conducting connections between the distal ends of the supply lines and the proximal ends of the lumina or an undesirable fluid-conducting transverse connection between the connection channels of the connecting unit can occur.

### SUMMARY

It is the object of the invention to provide a catheter arrangement of the type mentioned at the beginning and a method for producing such a catheter arrangement, the catheter arrangement and method having improved properties in relation to the prior art.

This object is achieved for the catheter arrangement in that the fluid-conducting connection between the supply line and the lumen is brought about by means of a fluid-tight joining connection between the distal end of the supply line and the proximal end of the lumen. By means of the solution according to the invention, a direct fluid-conducting connection between the supply line and the lumen is achieved. Accordingly, an otherwise customary, additional connecting unit can be dispensed with. The disadvantages and risks associated with such a connecting unit, in particular the formation of leakages or transverse connections between the connecting channels due to defective manufacturing, can to this extent be avoided. The fluid-tight joining connection can be designed in particular as a press connection, plug-in connection, screw connection, adhesive connection and/or welded connection between the distal end of the supply line and the proximal end of the lumen. For example, the distal end of the supply line can be plugged, pressed or screwed into the proximal end of the assigned lumen, or vice versa. Alternatively or additionally, the distal end of the supply line can be adhesively bonded and/or welded to the proximal end of the lumen. The fluid-tight joining connection is preferably nondetachable. Within the context of the invention, “proximal” denotes a position facing away from a catheter tip of the catheter arrangement. By contrast, “distal” refers to a position facing the catheter tip.

The solution according to the invention is suitable in a particularly preferred manner for a multi-lumen catheter arrangement having two or more lumina and accordingly two or more supply lines which are each assigned to one of the lumina. However, the solution according to the invention is not restricted to such multi-lumen catheter arrangements, but can also be used in a single-lumen catheter arrangement having just one lumen and one supply line.

In a refinement of the invention, the distal end of the supply line is plugged, in particular in a fluid-tight manner, into the proximal end of the lumen. If two lumina and two supply lines are provided, a first plug-in connection is formed between the first supply line and the first lumen and/or a second plug-in connection is formed between the second supply line and the second lumen. Said plug-in connections can be designed as such in a fluid-tight manner, for example by means of a corresponding adaptation of the outer diameters of the supply lines to the inner diameters of the lumina, thus resulting in each case in a fluid-tight press fit. Alternatively, the plug-in connections can be formed in a fluid-tight manner only to a limited extent, if at all, with it being possible for the distal ends of the supply lines to be welded and/or adhesively bonded in a fluid-tight manner to the proximal end of the respective lumen in order to faun the fluid-tightness of the joining connection. The same applies if just one plug-in connection is provided in a single-lumen catheter arrangement.

In a further refinement of the invention, the distal end of the supply line has an introducing portion which is integrally formed on the end side and is formed in a complementary manner with respect to a receiving portion, which is integrally formed on the end side, of the proximal end of the lumen. The introducing portion to of the supply line is integrally formed on the end side of the distal end and can be designed in particular as a pin, collar or nipple. The receiving portion is integrally Rained at the proximal end of the lumen and can be designed in particular in the manner of a sleeve or by means of a radial expansion of the lumen. The introducing portion is preferably integrally formed in one piece on the supply line. Accordingly, it is advantageous if the receiving portion is integrally formed in one piece on the catheter tube or on the lumen.

In a further refinement of the invention, the receiving portion is expanded, in particular thermally, in relation to a flow cross section of the lumen. A flow cross section of the lumen should be understood as meaning a cross section which is provided for guiding fluid. The flow cross section can be in particular round, oval, circular, semi-circular or sickle-shaped. The receiving portion can be formed in particular by means of pressing a suitable tool into the proximal ends of the lumen. If a multi-lumen catheter arrangement having at least two lumina and at least two supply lines is provided, the flow cross sections and therefore the receiving portions can be formed differently.

In a further refinement of the invention, the joining connection is welded, in particular in a fluid-tight manner. Accordingly, the joining connection between the distal end of the supply line and the proximal end of the lumen has a welded connection. The welded connection can be designed as such in a fluid-tight manner. For example, the distal end of the supply lines can be “butt-welded” to the proximal end of the lumen. Alternatively, the fluid-tight joining connection can have a plug-in connection in accordance with the statements above, with the welded connection additionally being provided. Both the supply line and the catheter tube are preferably manufactured from plastic at least in the region of the joining connection. The plastics composition of the supply lines here is coordinated with the plastics composition of the catheter tube in such a manner that they can be joined together by means of basically known plastics welding methods.

In a further refinement of the invention, a capsule unit is provided which is manufactured as a low-pressure injection moulded component made from plastic and is configured and/or arranged in such a manner that the region of the fluid-tight joining connection between the supply line and the lumen is encapsulated by means of the capsule unit. Manufactured as a low-pressure injection moulded component means that the capsule unit is produced using a manufacturing method primarily known up to now in the field of electrical engineering and which is also referred to as low-pressure moulding. The capsule unit surrounds the fluid-tight joining connection between the supply line and the lumen in the manner of a covering, a sheathing or a housing. The capsule unit preferably has at one end a conically formed contact portion which, in a correctly applied state of the catheter arrangement, is provided for contact with an entry point of the catheter tube on the patient. In addition, it is advantageous if the capsule unit has fixing portions by means of which the capsule unit and therefore the catheter arrangement can be fastened on the patient in a basically known manner.

In a further refinement of the invention, the capsule unit brings about an additional fluid sealing and/or tensile force relief of the fluid-tight joining connection. The fluid sealing brought about by means of the capsule unit is to this extent redundant. Accordingly, a leakage of the catheter arrangement can be avoided in the event of an unintentional detaching of the fluid-tight joining connection between the distal end of the supply line and the proximal end of the lumen. By means of this refinement of the invention, a particularly reliable sealing of the connection between the supply line and the catheter tube is accordingly achieved. If the capsule unit is manufactured as a low-pressure injection moulded component, said capsule unit is connected fixedly, in particular in an integrally bonded manner, to the supply line and to the catheter tube. A tensile force relief of the fluid-tight joining connection is thereby achieved. Accordingly, an undesirable detaching of the fluid-tight joining connection can be counteracted by means of the capsule unit. This is a particularly robust and patient-safe configuration of the invention.

In a further refinement of the invention, the capsule unit is manufactured from hot-melt adhesive, in particular from polyamide or polyolefin. Hot-melt adhesives are basically known as such and have a thermoplastic composition. Hot-melt adhesives are preferably suitable for processing by means of low-pressure injection moulding since they have a comparatively low viscosity in the molten state. This makes it possible for processing to take place at low pressures—in comparison to conventional plastics injection moulding. A pressure-induced deformation of the fluid-tight joining connection during the formation of the capsule unit can thereby be avoided.

In a further refinement of the invention, the catheter arrangement is a central venous catheter arrangement or a peripherally inserted catheter arrangement or a midline catheter arrangement.

The object on which the invention is based is achieved for a method of the type mentioned at the beginning in that a step is provided: joining the distal end of the supply line together with the proximal end of the lumen in a fluid-tight manner. By means of the solution according to the invention, a complicated and potentially error-prone production of the fluid-tight connection by means of an otherwise customary connecting unit can be dispensed with. Instead, the distal end of the supply line is joined, preferably directly, together with the proximal end of the lumen, for example by means of a press connection, plug-in connection, screw connection, adhesively bonding connection and/or welded connection. Accordingly, for the production of a multi-lumen catheter arrangement, the distal end of the respective supply line is joined together with the proximal end of the respective lumen in a fluid-tight manner.

In a further refinement of the invention, the method has a step: integrally forming an end-side introducing portion on the distal end of the supply line. The integral forming of the introducing portion preferably takes place by means of deformation of the distal end of the supply line. The introducing portion is preferably integrally formed here in a complementary manner to a cross-sectional shape of the lumen, for example in a round, oval, circular or semi-circular, crescent-shaped or sickle-shaped manner. Such integral forming of the introducing portion permits a simple and accurately fitting joining of the supply line together with the lumen.

In a further refinement of the invention, the method has a step: integrally forming an end-side receiving portion on the proximal end of the lumen by means of radial expansion of the lumen. The receiving portion is preferably integrally formed on the proximal end of the lumen by means of deformation. For example, a correspondingly configured tool in the form of a mandrel, a pin or the like can be pressed into the proximal end of the lumen such that the latter is plastically radially expanded. The receiving portion is preferably shaped substantially conically. If the supply line is provided with a corresponding introducing portion, it is advantageous if the configuration of the receiving portion is coordinated with the configuration of the introducing portion in such a manner that a transition fit or press fit arises therebetween.

In a further refinement of the invention, the integral forming of the introducing portion and/or of the receiving portion takes place by means of deformation under the action of pressure and/or heat. Both the supply line and the catheter tube are preferably manufactured from thermoplastic. Thermoplastics soften under the action of heat and, in a softened state, are deformable by application of comparatively small pressure or deformation forces. This configuration of the invention permits the introducing and/or receiving portions to be able to be produced particularly simply and cost-effectively.

In a further refinement of the invention, during the joining together in a fluid-tight manner the introducing portion of the supply line is plugged into the receiving portion of the lumen. Accordingly, a direct force- and/or form-fitting connection is produced between the distal ends of the supply line and the proximal end of the lumen. The fluid tightness of the joining connection can already be brought about here by plugging of the introducing portion into the receiving portion as such. For example, by the outer contour of the introducing portion being coordinated with the inner contour of the receiving portion in such a manner that a press fit between the supply line and the respective lumen, and therefore fluid tightness, results. Alternatively, or additionally, after the introducing portion is plugged into the receiving portion, the fluid tightness of the joining connection can be brought about by means of further method steps, for example adhesive bonding, welding or the like.

In a further refinement of the invention, during the joining together in a fluid-tight manner, the distal end of the supply line is welded to the proximal end of the lumen, in particular the introducing portion is welded to the receiving portion. If the supply line and the catheter tube are manufactured from thermoplastic, the distal end of the supply line can be connected to the proximal end of the lumen by means of basically known plastics welding methods, for example by means of circular welding, laser transmission welding, induction welding or ultrasonic welding.

In a further refinement of the invention, the method has a step: testing, in particular by means of computer-aided image recognition and image processing, an assignment of the introducing portion to the receiving portion and/or an assignment of the distal end of the supply line to the proximal ends of the lumen. In order to be able to ensure detection and/or identification of the respective portions and/or ends by means of computer-aided image processing, it is advantageous if the portions and/or the ends are provided with corresponding marking elements, for example a coloured and/or graphical marking. Testing by means of computer-aided image processing means that, in order to avoid a defective assignment of the portions and/or ends and therefore for quality assurance, use is made of a method which is basically known as such in the sphere of automation technology and which is also referred to as machine vision or image understanding. Such testing of the assignment of the portions and/or of the ends can take place in particular during the production of a multi-lumen catheter arrangement, for example before the introducing portions are plugged into the respective receiving portion. Alternatively or additionally, the testing of the assignment can take place before the welding of the supply lines to the catheter tube or of the introducing portions to the respective receiving portion. In addition, it is advantageous if, during the testing, the tightness of the joining connection between the distal end of the respective supply line and the proximal end of the respective lumen is also checked. For this purpose, the computer-aided image recognition and image processing can be configured in such a manner that a defect at the joining connection, for example an incompletely formed welded connection, can be detected.

In a further refinement of the invention, the method has a step: encapsulating the fluid-tight joining connection between the supply line and the lumen by means of low-pressure plastics injection moulding, with a capsule unit being formed. Low-pressure plastics injection moulding is basically known in particular in the sphere of producing electric and/or electronic components. It is also referred to as low-pressure moulding. In this method, a thermoplastic hot-melt adhesive, for example polyamide or polyolefin, is liquefied under the action of heat. For example, the hot-melt adhesive is heated to a temperature of 180° C. to 240° C., preferably 210° C. The hot-melt adhesive liquefied in such a manner is pressed into a corresponding injection mould at a comparatively low pressure, preferably within the range of 3.5 bar to 14 bar. On account of such low injection pressures, low-pressure moulding is preferably suitable for encapsulating sensitive components which could be deformed and/or damaged under the action of relatively high pressures, such as prevail, for example, during conventional plastics injection moulding. Accordingly, an otherwise customary supporting of the lumen and of the supply line by means of what is referred to as an insert-moulding needle can be dispensed with. This refinement of the invention makes it possible in particular to achieve improved sealing of the fluid-tight joining connection and tensile force relief between the supply line and the catheter tube.

### BRIEF DESCRIPTION OF THE DRAWING FIGURES

Further advantages and features of the invention emerge from the description below of preferred exemplary embodiments of the invention that are illustrated with reference to the drawings.

FIG. 1 shows, in a schematic illustration, an embodiment of a catheter arrangement according to the invention comprising a catheter tube and two catheter supply lines,

FIG. 2 shows, in a schematic cross-sectional illustration, the catheter tube of the catheter arrangement along an intersecting line A-A according to FIG. 1,

FIG. 3 shows, in a schematic longitudinal sectional illustration, the catheter arrangement according to FIGS. 1 and 2 in the region of a fluid-tight joining connection between the supply lines and the lumen of the catheter tube,

FIG. 4 shows, in a schematic perspective illustration, a proximal region of the catheter tube of the catheter arrangement according to FIGS. 1 to 3,

FIG. 5 shows, in a schematic perspective illustration, a distal region of one of the supply lines of the catheter arrangement according to FIGS. 1 to 4,

FIG. 6 shows, in a schematic flow illustration, an embodiment of a method according to the invention for producing the catheter arrangement according to FIGS. 1 to 5,

FIG. 7 shows, in a schematic flow illustration, details of a method step of the method according to FIG. 6,

FIG. 8 shows, in a schematic flow illustration, details of a further method step of the method according to FIG. 6, and

FIG. 9 shows, in a schematic flow illustration, details of a further method step of the method according to FIG. 6.

### DETAILED DESCRIPTION

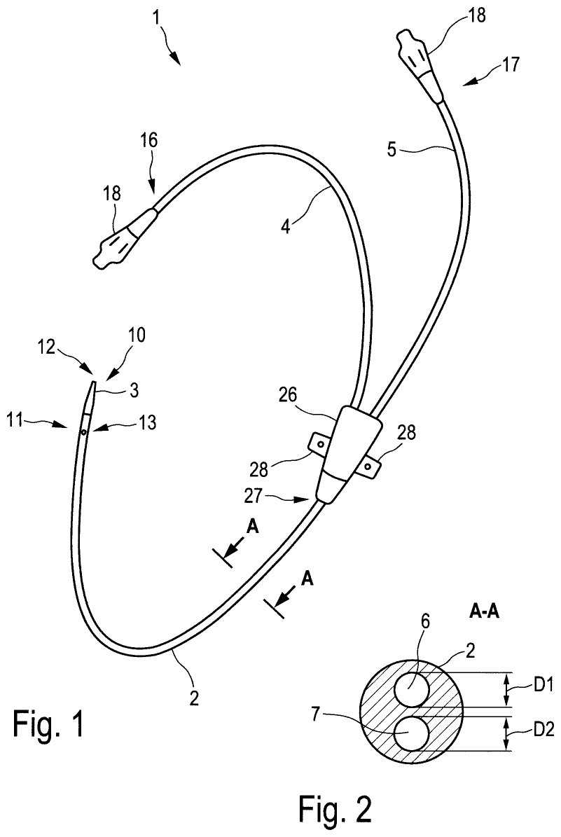

A catheter arrangement 1 according to FIG. 1 is provided for use in infusion therapy. The catheter arrangement 1 is designed in the form of a two-lumen central venous catheter arrangement. Irrespective thereof, the solution according to the invention can, of course, also be used in the case of single-lumen, three-lumen or multi-lumen catheter arrangements and is not restricted to central venous catheter arrangements, but rather in particular can also be used in a peripherally inserted catheter arrangement or a midline catheter arrangement.

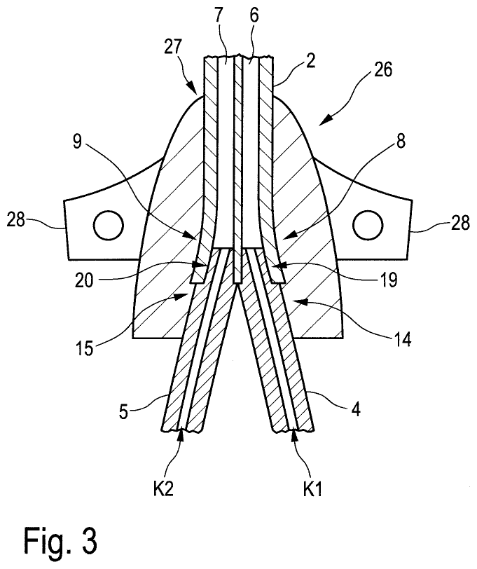

The catheter arrangement 1 has a catheter tube 2 with a catheter tip 3 and two catheter supply lines 4, 5 which are referred to below as first supply line 4 and second supply line 5. Both the catheter tube 2 and the supply lines 4, 5 are manufactured from plastic and are flexurally flexible. As is apparent in particular with reference to FIG. 2, the catheter tube 2 has a first lumen 6 and a second lumen 7. The lumina 6, 7 extend separated from each other through the catheter tube 2 and each have a proximal end 8, 9 (FIG. 3) and a distal end 10, 11. The distal end 10 of the first lumen 6 leads into an outlet opening 12 of the catheter tip 3. The distal end 11 of the second lumen 7 leads into a radial opening 13 of the catheter tube 2. Furthermore, the supply lines 4, 5 each have a distal end 14, 15 (FIG. 3) and a proximal end 16, 17. Both the proximal end 16 of the first supply line 4 and the proximal end 17 of the second supply line 5 are provided with a respective connection element 18. The connection elements 18 are each provided for the fluid-conducting connection of the supply lines 4, 5 to an infusion system (not specifically apparent), in particular an infusion container or the like. For this purpose, the connection elements 18 can each be provided with a Luer connection which is known as such. As is apparent with reference to FIG. 3, the distal end 14 of the first supply line 4 is connected in a fluid-conducting manner to the proximal end 8 of the first lumen 6 and the distal end 15 of the second supply line 5 is connected in a fluid-conducting manner to the proximal end 9 of the second lumen 7. Two channels K1, K2 which are separate from each other are thereby formed. The channels K1, K2 extend from the proximal end 16, 17 of the respective supply line 4, 5 as far as the distal end 10, 11 of the lumen 6, 7 assigned to the respective supply line 4, 5. This permits, for example, a simultaneous administration of incompatible medicinal fluids by means of the catheter arrangement 1, with mixing of the fluids within the catheter arrangement 1 being avoided.

As is furthermore apparent in particular with reference to FIG. 3, the fluid-conducting connections between the supply lines 4, 5 and the lumina 6, 7 are brought about by means of a respective fluid-tight joining connection 19, 20 between the distal end 14, 15 of the respective supply line 4, 5 and the proximal end 8, 9 of the respective lumen 6, 7. For this purpose, the distal end 14 of the first supply line 4 is plugged into the proximal end 8 of the first lumen 6, and the distal end 15 of the second supply line 5 is plugged into the proximal end 9 of the second lumen 7. The plug-in connections between the supply lines 4, 5 and the respective lumen 6, 7 are configured here in such a manner that the joining connections 19, 20 are fluid-tight. For this purpose, a press fit is in each case provided between the distal ends 14, 15 of the supply lines 4, 5 and the respective proximal end 8, 9 of the lumina 6, 7.

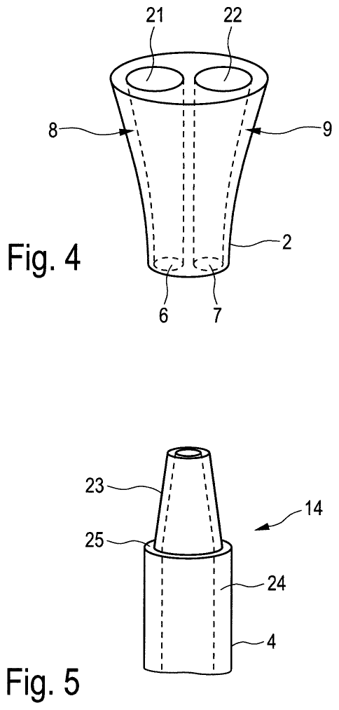

Further details of the fluid-tight joining connections 19, 20 are apparent in particular with reference to FIGS. 4 and 5. As is apparent with reference to FIG. 4, a receiving portion 21, 22 is in each case integrally formed on the end side of the proximal ends 8, 9 of the lumina 6, 7. In comparison to FIG. 2, it is apparent that the receiving portions 21, 22 are radially expanded in relation to a flow cross section D1, D2 of the respective lumen 6, 7. In addition, the distal ends 14, 15 of the supply lines 4, 5 are each provided with an introducing portion. FIG. 5 shows the first supply line 4 provided with an introducing portion 23. The second supply line 5 is configured in a corresponding manner in the region of its distal end 15, and therefore only the introducing portion 23 of the first supply line 4 will be described in detail with reference to FIG. 5 in order to avoid repetitions. The introducing portion 23 is integrally formed on the end side of the distal end 14 of the first supply line 4 and is configured in a complementary manner with respect to the receiving portion 21 of the proximal end 8 of the first lumen 6. In this respect, the introducing portion 23 has a conical outer contour and, in addition, is offset radially in relation to a circumferential surface 24 of the first supply line 4 such that a radial collar 25 is arranged between the introducing portion 23 and the further axial profile of the first supply line 4. However, said radial collar 25 does not absolutely have to be present. Instead, the region can be configured, for example, conically or cylindrically. It can be seen with reference to FIG. 3 that the distal end 14 of the first supply line 4 is plugged into the proximal end 9 of the first lumen 6 in such a manner that the radial collar 25 comes into contact on the end side of the catheter tube 2, and therefore the supply line 4 is fixed in the axial direction in relation to the catheter tube 2. The same applies here to the plug-in connection between the second supply line 5 and the second lumen 7, but this does not necessarily have to be the case.

The receiving portions 21, 22 are designed here in the form of an inner cone in each case, wherein the cross sections of the receiving portions 21, 22 correspond in their basic shape to the flow cross sections D1, D2 and in this respect are of circular design. Of course, it is also possible for a differing configuration of the flow cross sections and of the receiving portions to be provided. For example, the flow cross sections of the lumina can be configured in an oval, semi-circular, crescent-shaped or sickle-shaped manner. In such a case, the receiving portions have an accordingly designed shape. In such a case, the introducing portions of the supply lines are accordingly also adapted in their shape.

The above-described plug-in connections between the supply lines 4, 5 and the respective lumen 6, 7 are designed here in a fluid-tight manner as such, and therefore the fluid tightness of the joining connections 19, 20 is already produced by the configuration of said plug-in connections. The joining connections 19, 20 are additionally welded. Should the plug-in connections between the supply lines 4, 5 and the respective lumen 6, 7 not already be designed in a fluid-tight manner as such, the fluid tightness of the joining connections 19, 20 can also be brought about solely by welding of the distal ends 14, 15 of the supply lines 4, 5 to the proximal end 8, 9 of the respective lumen 6, 7.

As is furthermore apparent with reference to FIG. 1, the catheter arrangement 1 also has a capsule unit 26. The capsule unit 26 is manufactured as a low-pressure injection moulded component made from plastic and is configured and/or arranged in such a manner that the region of the fluid-tight joining connections 19, 20 between the supply lines 4, 5 and the lumina 6, 7 is encapsulated by means of the capsule unit 26. This is also apparent with reference to FIG. 3. The capsule unit 26 as such does not form a fluid-guiding portion of the channels K1, K2. Instead, the capsule unit 26 brings about an additional fluid sealing of the fluid-tight joining connections 19, 20. In addition, a tensile force relief of the fluid-tight joining connections 19, 20 is brought about by means of the capsule unit 26, by the capsule unit 26 firstly being fixedly connected to the supply lines 4, 5 and secondly being fixedly connected to the catheter tube 2. The capsule unit 26 is manufactured from a hot-melt adhesive. Hot-melt adhesives which can be processed by means of low-pressure injection moulding are basically known. For example, polyamide or polyolefin can be used as the hot-melt adhesive. As is furthermore apparent with reference to FIG. 1 and FIG. 3, the capsule unit 26 has a contact portion 27 which has a substantially conical basic shape. In a correctly applied state of the catheter arrangement 1, the contact portion 27 is provided to enter into contact with an entry point of the catheter tube 2 on the patient. As a result of the conical configuration of the contact portion 27, bleeding at the above-described entry point of the catheter tube 2 can be counteracted. In addition, the capsule unit 26 has two fixing portions 28. The fixing portions 28 are arranged on opposite sides of the capsule unit 26. The capsule unit and therefore the catheter arrangement 1 can be fixed on the patient in a basically known manner by means of the fixing portions 28.

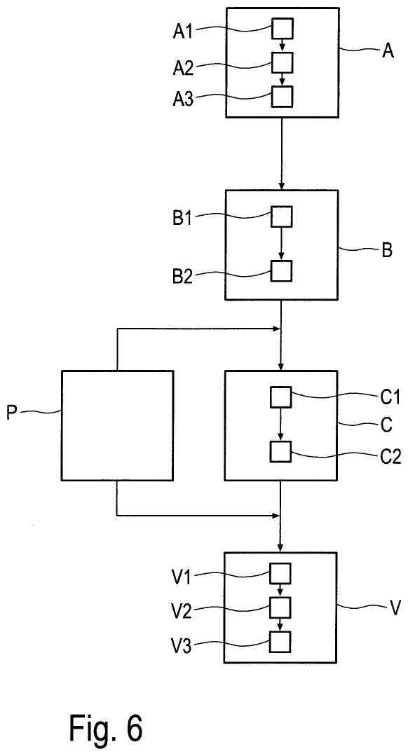

An embodiment of a method according to the invention according to FIGS. 6 to 9 is provided for producing a catheter arrangement 1 according to FIGS. 1 to 5. The method is based here on the fact that both the supply lines 4, 5 and the catheter tube 2 are present in the form of basically known plastics semi-finished products.

The method first of all provides a step: (A) integrally forming an end-side introducing portion (23, cf. FIG. 5) in each case on the distal end 14 of the first supply line 4 and on the distal end 15 of the second supply line 5.

The method then provides a further step: (B) integrally forming an end-side receiving portion (21, 22, cf. FIG. 4) in each case on the proximal end 8 of the first lumen 6 and on the proximal end 9 of the second lumen 7 by means of radial expansion of the first lumen 6 and of the second lumen 7.

The method then provides a further step: (C) joining the distal ends 14, 15 of the supply lines 4, 5 together with the proximal end 8, 9 of the respective lumen 6, 7 in a fluid-tight manner. In this step, the distal end 14 of the first supply line 4 is joined together with the proximal end 8 of the first lumen in a fluid-tight manner and the distal end 15 of the second supply line 5 is joined together with the proximal end 9 of the second lumen 7 in a fluid-tight manner. If the lumina 6, 7 are provided at their proximal ends 8, 9 with a respective receiving portion 21, 22 and the supply lines 4, 5 are each provided at their distal ends 14, 15 with an introducing portion—but this is not absolutely necessary—in this step the receiving portions 21, 22 are joined together with the introducing portion of the respective lumen 6, 7 in a fluid-tight manner.

Furthermore, temporally before and/or after the fluid-tight joining together (C), the method provides a step: (P) testing an assignment of the introducing portions (23, cf. FIG. 5) to the receiving portions 21, 22 and/or an assignment of the distal ends 14, 15 of the supply lines 4, 5 to the proximal ends 8, 9 of the lumina 6, 7. It is tested in this step whether the first supply line 4 is assigned to the first lumen 6 and the second supply line 5 to the second lumen 7. An erroneous assignment which may lead, for example, to an impairment of the function of the catheter arrangement 1 or to leakiness of the fluid-tight joining connections 19, 20 is thereby avoided. The testing step (P) provides the use of a basically known method of computer-aided image recognition and image processing, which is also referred to as mechanical vision or image understanding.

After the fluid-tight joining together (C) or optionally the testing (P), the method provides a further step: (V) encapsulating the fluid-tight joining connections 19, 20 between the supply lines 4, 5 and the lumina 6, 7 by means of low-pressure plastics injection moulding, with a capsule unit (26, cf. FIGS. 1, 3) being formed.

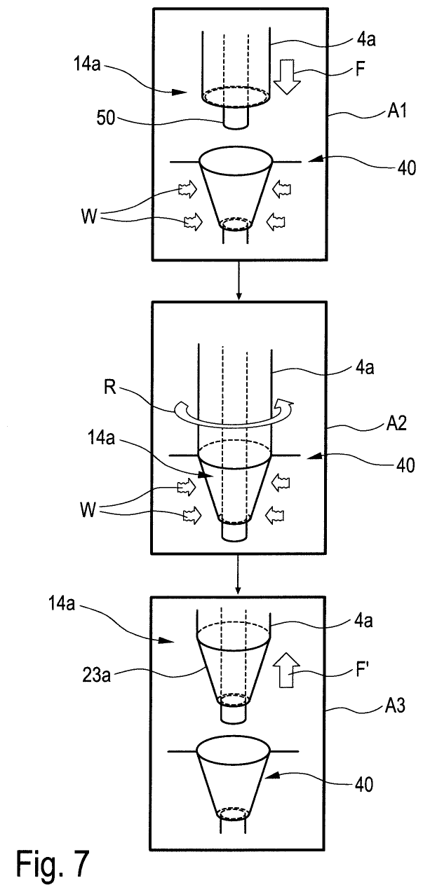

Further details of the method according to FIG. 6 are apparent with reference to FIGS. 7 to 9. Said figures refer to details of the above-described steps (A), (B), (C) and (V) of the method and also clarify said details illustratively with reference to a further embodiment of a catheter arrangement according to the invention. This catheter arrangement substantially corresponds to the embodiment described previously with reference to FIGS. 1 to 5. The catheter arrangement which is apparent at any rate in regions with reference to FIGS. 7 to 9 essentially differs from the catheter arrangement 1 according to FIGS. 1 to 5 in that this catheter arrangement has three lumina and in this respect is provided with three supply lines and three lumina instead of with two supply lines and two lumina (cf. FIG. 1). In order to avoid repetitions, reference is therefore made to the disclosure with regard to the embodiment according to FIGS. 1 to 5. Functionally identical and/or structurally identical portions and parts of the catheter arrangement apparent at any rate in regions with reference to FIGS. 7 to 9 are provided with the same reference signs with the addition of the letter a.

Details of the integrally forming method step (A) are apparent with reference to FIG. 7. In a first partial step (A1), a supply line 4a is pressed with its distal end 14a into a mould 40 under the action of pressure—which is intended to be clarified by means of the arrow F. The mould 40 has a conical mould contour and is heated, which is intended to be clarified with reference to the arrows W. The distal end 14a is supported on its inner diameter by means of a mandrel 50. The mandrel 50 prevents the supply line 4a from being closed on the end side as it is being pressed into the mould 40.

In a further partial step (A2), the distal end 14a of the supply line 4a is pressed into the mould 40 in such a manner that it comes into contact on the end side with the bottom region of the mould 40. In addition, the supply line 4a is rotated about its longitudinal axis and the action of heat W is maintained. An introducing portion 23a is thereby formed at the distal end 14a of the supply line 4a.

In a further partial step (A3), the supply line 4a is demoulded in the axial direction from the mould 40. This is clarified with reference to the arrow F′.

Depending on the number of provided supply lines, the above-described partial steps (A1-A3) are repeated for each of the provided supply lines.

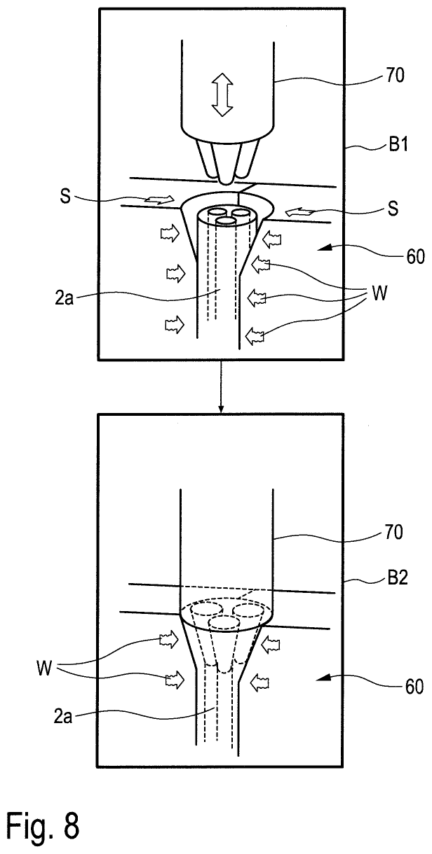

Further details of the integrally forming step (B) of the method according to FIG. 6 are clarified with reference to FIG. 8. A first partial step (B1) makes provision for the proximal end of a catheter tube 2a, which is configured with three lumina, to be clamped in a mould 60 having two partial halves. The clamping movement of the two partial halves of the mould 60 is intended to be clarified with reference to the arrows S. In addition, the mould 60 is under the action of heat W. The catheter tube 2a is thereby softened in the region of clamping between the two partial halves. In an upper region with respect to the plane of the drawing of FIG. 8, the mould 60 has a conical expansion, and therefore the catheter tube 2a is first of all spaced apart from the mould 60 in this region. Furthermore, the partial step (B1) makes provision for a punching tool 70 to be pressed in the axial direction into the proximal ends of the lumina (not denoted specifically) of the catheter tube 2a. The punching tool 70 is provided on the end sides with conically configured pins which, during the axial movement of the punching tool 70, enter the proximal ends of the lumina and thereby expand the latter in the radial direction.

In a further partial step (B2), the punching tool 70 is completely moved downward in the axial direction, with the end-side pins of the punching tool 70 completely entering the proximal ends of the lumina of the catheter tube 2a. Receiving portions which are not apparent specifically (cf. FIGS. 4, 21, 22) are thereby formed at the proximal ends of the lumina.

In a further partial step which is not apparent specifically, the punching tool 70 is moved upward in the axial direction and the catheter tube 2a is unclamped from the two partial halves of the mould 60 and removed from the mould 60 for further handling.

During the fluid-tight joining together (C), the introducing portions formed in such a manner are then plugged into the receiving portions of the lumina (C1) and welded to one another (C2), which is clarified schematically with reference to FIG. 6.

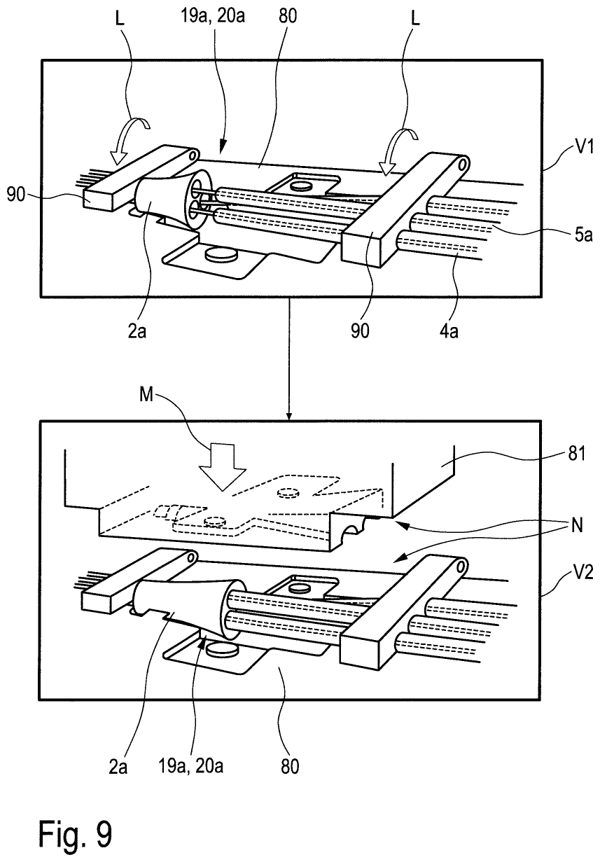

Further details of the encapsulating step (V) of the method according to FIG. 6 are apparent with reference to FIG. 9. In a first partial step (V1), the catheter tube 2a is placed together with the first supply line 4a, a second supply line 5a and a further third supply line (not denoted specifically) into a lower partial half 80 of a low-pressure injection mould. In this state, fluid-tight joining connections 19a, 20a between the distal ends of the respective supply lines 4a, 5a and the proximal end of the respective lumen of the catheter tube 2a are already formed. The same applies to the third supply line (not denoted specifically) which is joined together with the proximal end of the lumen, assigned thereto, of the catheter tube 2a in a likewise fluid-tight manner. As is apparent with reference to FIG. 6, in order to form the fluid-tight joining connections 19a, 20a, the supply lines 4a, 5a are plugged into the respective lumen of the catheter tube 2a (C1) and/or are welded to said lumen (C2). The position of the catheter tube 2a and of the supply lines 4a, 5a and of the further supply line (not denoted specifically) is fixed in relation to the lower partial half 80 of the low-pressure injection mould by means of clamping jaws 90. This is clarified with reference to the arrows L.

In a further partial step (V2), it is provided that an upper partial half 81 of the low-pressure injection mould is lowered onto the lower partial half 80, which is clarified with reference to the arrow M, with the low-pressure injection mould being completely closed. The partial halves 80, 81 here form a negative shape N of a capsule unit, which is not apparent specifically (cf. FIGS. 1, 26). After the complete closing of the two partial halves 80, 81, the negative shape N is filled in a basically known manner with molten hot-melt adhesive under pressurization within a range of approximately 3.5 bar-14 bar. A capsule unit encapsulating the fluid-tight joining connections 19a, 20a between the supply lines 4a, 5a and the lumina of the catheter tube 2a is thereby formed.

In a further partial step (V3) (cf. FIG. 6), the capsule unit formed in such a manner is demoulded in a basically known manner from between the partial halves 80, 81 of the low-pressure injection mould, and the catheter arrangement is removed.

1. A catheter arrangement comprising a catheter tube with a lumen, wherein the lumen has a proximal end and a distal end, and a supply line, wherein the supply line has a distal end and a proximal end, and wherein the distal end of the supply line is connected in a fluid-conducting manner to the proximal end of the lumen with a fluid-conducting connection, wherein the fluid-conducting connection between the supply line and the lumen is brought about by a fluid-tight joining connection between the distal end of the supply line and the proximal end of the lumen.

2. The catheter arrangement according to claim 1, wherein the distal end of the supply line is plugged into the proximal end of the lumen.

3. The catheter arrangement according to claim 1, wherein the distal end of the supply line has an introducing portion which is integrally formed on an end side of the distal end, and is formed in a complementary manner with respect to a receiving portion, which is integrally formed on an end side of the proximal end of the lumen.

4. The catheter arrangement according to claim 3, wherein the receiving portion is expanded in relation to a flow cross section of the lumen.

5. The catheter arrangement according to claim 1, wherein the joining connection is welded.

6. The catheter arrangement according to claim 1, wherein a capsule unit is provided which is manufactured as a low-pressure injection molded component made from plastic and is configured and/or arranged in such a manner that the region of the joining connection between the supply line and the lumen is encapsulated by the capsule unit.

7. The catheter arrangement according to claim 6, wherein the capsule unit brings about an additional fluid sealing and/or tensile force relief of the fluid-tight joining connection.

8. The catheter arrangement according to claim 6, wherein the capsule unit is manufactured from hot-melt adhesive.

9. The catheter arrangement according to claim 1, wherein the catheter arrangement is a central venous catheter arrangement or a peripherally inserted central catheter arrangement or a midline catheter arrangement.

10. A method for producing a catheter arrangement according to claim 1, the method comprising: joining the distal end of the supply line together with the proximal end of the lumen in a fluid-tight manner.

11. The method according to claim 10, further comprising the step of integrally forming an end-side introducing portion onto the distal end of the supply line.

12. The method according to claim 11, having a step: (B) further comprising the step of integrally forming an end-side receiving portion onto the proximal end of the lumen by radial expansion of the lumen.

13. The method according to claim 12, wherein the integral forming of the introducing portion and/or of the receiving portion takes place by deformation under pressure and/or heat.

14. The method according to claim 12, wherein, during joining the distal end of the supply line together with the proximal end of the lumen in a fluid-tight manner, the introducing portion of the supply line is plugged into the receiving portion of the lumen.

15. The method according to claim 10, wherein, during joining the distal end of the supply line together with the proximal end of the lumen in a fluid-tight manner, the distal end of the supply line is welded to the proximal end of the lumen.

16. The method according to claim 14 further comprising the step of testing an assignment of the introducing portion to the receiving portion and/or an assignment of the distal end of the supply line to the proximal end of the lumen.

17. The method according to claim 10 further comprising the step of encapsulating the joining connection between the supply line and the lumen by low-pressure plastics injection molding, with a capsule unit being formed.
